(19) United States
(12) Patent Application Publication
(10) Pub. No.: US 2010/0145425 A1
(43) Pub. Date: Jun. 10, 2010

(54) ELECTRODE FOR STIMULATING CRANIAL NERVES AND SUBSTRATE COMPRISING THE SAME

(75) Inventors: Sangdon Jung, Daejeon-city (KR); Myungae Chung, Daejeon-city (KR); Sanghyeob Kim, Daejeon-city (KR); Gookhwa Kim, Jeonbuk (KR); Ikhyun Kim, Daejeon-city (KR)

Correspondence Address: STAAS & HALSEY LLP, SUITE 700, 1201 NEW YORK AVENUE, N.W., WASHINGTON, DC, 20005, US

(73) Assignee: Electronics and Telecommunications Research Institute, Daejeon-city (KR)

(21) Appl. No.: 12/634,247

(22) Filed: Dec. 9, 2009

(30) Foreign Application Priority Data
Dec. 10, 2008 (KR) 10-2008-0125473

(51) Int. Cl. A61N 1/05; A61N 1/04

(52) U.S. Cl. 607/116; 607/115

(57) ABSTRACT

An electrode for stimulating cranial nerves comprising a glass fiber having first and second holes, and a metal wire disposed in the first hole.

### RELATED APPLICATIONS

The present application claims priority to Korean Patent Application Serial Number 10-2008-0125473, filed on Dec. 10, 2008, the entirety of which is hereby incorporated by reference.

### BACKGROUND OF THE INVENTION

1. Field of the Invention

The present invention relates to an electrode for stimulating cranial nerves and a substrate comprising the same. More specifically, the present invention relates to an electrode for stimulating cranial nerves comprising a glass fiber and a metal wire, and a substrate comprising the electrode.

2. Description of the Related Art

Generally, stimulating cranial nerves or nerve cells with electricity is very useful for treating strokes, preventing attacks, alleviating pain, performing remote control of animal, etc. Electrodes providing the electric stimulus are classified into mono-polar and bi-polar electrodes. The bi-polar electrode has the characteristic of being capable of minimizing a stimulus applied to unnecessary regions because the range of the stimulus is geometrically restricted.

FIG. 1 is a perspective view illustrating an electrode for stimulating cranial nerves using a conventional bi-polar electrode.

In FIG. 1, the conventional electrode 10 which stimulates a cranial nerve has the structure in which two thin metal wires 111 and 112 are sealed with a bio-stable polymer 120 such as epoxy, polyimide, or Teflon.

However, the conventional electrode for stimulating a cranial nerve has a small selection of bio-stable polymers which are applicable thereto. Further, even after application of the bio-stable polymer, the electrode has poor mechanical stability and poor machinability. For the reason, it is difficult to integrate and miniaturize such electrodes. Thus, the conventional electrode for stimulating cranial nerves is not suitable for the implementation of an embedded electric stimulating system, which essentially requires the integration and miniaturization of an electrode.

Meanwhile, generally, a device or an electrode inserted into the brain is considered as a foreign substance by spongiocytes. The spongiocytes then secrete protein fibers around the electrode so as to insulate it; the protein fibers serve as an insulator, which results in an increase in resistance, reducing the electrical stimulation effect.

A general solution proposed to solve the problem of the reduction in the electrical stimulation effect is to, upon insertion of an electrode, supply drugs such as a growth factor or the like to thereby restrict the function of the spongiocytes. To this end, the University of Michigan developed a method of formation of a micro tube, which can supply chemicals to a semiconductor substrate, using a Micro-Electro-Mechanical-System (MEMS) process. However, semiconductor based machining using the MEMS process has problems in that the machining cost is expensive and in that the semiconductor substrate is harmful to the human body and thus it should be separately sealed with a bio-stable polymer or the like.

### SUMMARY OF THE INVENTION

Accordingly, the present invention has been made keeping in mind the above problems occurring in the related art. An object of the present invention is to provide an electrode for stimulating cranial nerves, which can employ metal wires having a diversity of shapes and into which the metal wires can be easily inserted.

Further, another object of the present invention is to provide an electrode for stimulating cranial nerves, to which a machining method such as mechanical polishing, welding or the like is applicable and which can with ease be additionally machined.

Furthermore, another object of the present invention is to provide an electrode for stimulating cranial nerves, which can ensure a micro tube using a simple process and can supply drugs to the nerve system at low cost.

In order to achieve the object, the present invention provides an electrode for stimulating cranial nerves comprising: a glass fiber having first and second holes; and a metal wire disposed in the first hole.

Further, the present invention provides a substrate comprising a glass substrate having at least one electrode insertion area; electrodes each being disposed in the electrode insertion area and including a glass fiber having first and second holes, and a metal wire disposed in the first hole; and a connector connecting the glass substrate and the electrodes together.

According to the present invention, the electrode for stimulating cranial nerves can employ a diversity of shapes of metal wire which can be easily inserted. The electrode can employ a machining method such as mechanical polishing, welding, or the like, and can be additionally machined with ease. Further, according to the present invention the electrode can ensure the micro tube using a simple process so as to supply drugs to the nerve system at a low cost.

### BRIEF DESCRIPTION OF THE DRAWINGS

The above and other objects, features and advantages of the present invention will be more clearly understood from the following detailed description when taken in conjunction with the accompanying drawings, in which:

FIG. 1 is a perspective view illustrating a conventional electrode for stimulating cranial nerves;

FIG. 2 is a perspective view illustrating an electrode for stimulating cranial nerves according to an embodiment of the present invention; and

FIG. 3 is a perspective view illustrating a substrate according to another embodiment of the present invention.

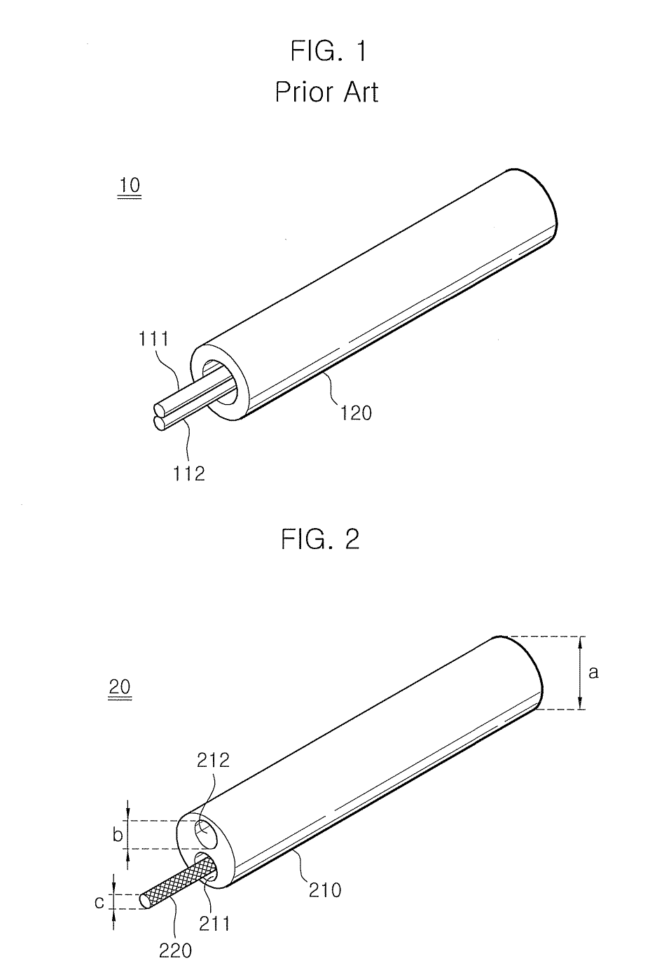

### DETAILED DESCRIPTION OF THE INVENTION

Reference will now be made in greater detail to a preferred embodiment of the invention, an example of which is illustrated in the accompanying drawings.

I. Electrode for Stimulating Cranial Nerves

FIG. 2 is a perspective view illustrating an electrode for stimulating cranial nerves according to an embodiment of the present invention.

Referring to FIG. 2, the electrode 20 for stimulating cranial nerves comprises a glass fiber 210 and a metal wire 220.

The electrode 20 for stimulating cranial nerves has a diameter in the range of preferably 100 μm to 290 μm, more preferably 150 μm to 250 μm. If the diameter satisfies the above-mentioned range, it is advantageous in that an electrode implements the integration and miniaturization, which are essential elements for an embedded electrode for stimulating cranial nerves.

The glass fiber 210 has first and second holes 211 and 212, and the metal wire 220 is disposed in the first hole 211. The second hole 212 is a hole for supplying a nervous-system drug to cranial nerves, and it is preferable that the hole is a hollow hole. The first hole 211 may have any shape but preferably that of a circle or an oval, so long as the metal wire 220 can be disposed in the first hole. Similarly, the second hole 212 may have any shape but preferably that of a circle or an oval, so long as it can supply a drug to the nerve therethrough. The first and second holes 211 and 212 have preferably the diameters b in the range of 20 μm to 100 μm. If the diameters are in the stated range, upon insertion of an electrode into a fine cranial nerve, the electrode can be firmly fixed. Further, it is advantageous in that an electrode implements the integration and miniaturization which are essential elements for an embedded electrode for the stimulation of cranial nerves.

If needed, the glass fiber 210 may further include one or more holes having a metal wire or supplying a nervous-system drug.

The metal wire 220 disposed in the first hole 211 is a bi-polar electrode, preferably. The shape of the metal wire 220 is not particularly restricted, but may be of a circle or an oval, so long as it can stimulate cranial nerves and can be disposed in the first hole. Further, a diameter c of the metal wire 220 is not particularly restricted, but may be preferably in the range of 15 μm to 90 μm, so long as it is fittable within the first hole 211 in the range. It is advantageous for an electrode to be applied to fine cranial nerves, if the diameter satisfies the above-mentioned range. Further, it is also advantageous in implementing the integration and miniaturization of an electrode, which are essential elements for an embedded electrode for stimulating cranial nerves.

The electrode 20 for the stimulating cranial nerves of the present invention can use heat- or light-curable polymer (not shown) in order to fix the glass fiber 210 and the metal wire 220 together. The polymer may be heat-curable or UV-curable epoxy, preferably.

II. Substrate comprising Electrode for stimulating Cranial Nerves

The present invention also provides a substrate comprising the electrode for stimulating cranial nerves.

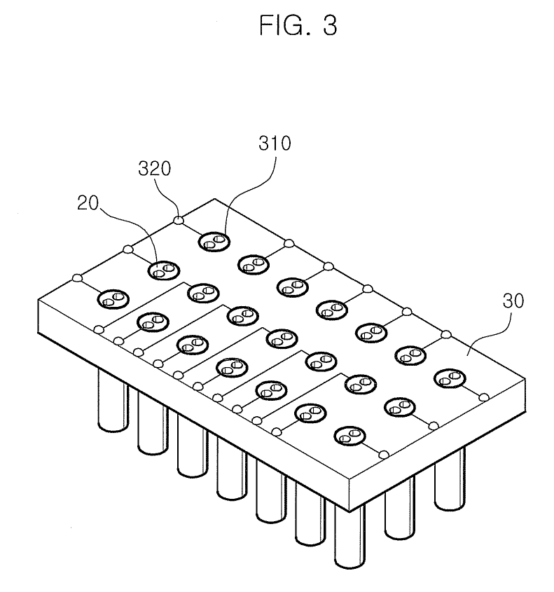

FIG. 3 is a perspective view illustrating the substrate having an electrode for stimulating cranial nerves according to another embodiment of the present invention.

Referring to FIG. 3, the substrate includes a glass substrate 30 having at least one electrode insertion areas 310 and the electrodes 20, as explained with reference to FIG. 2, each being disposed in the electrode insertion area 310. The substrate 30 and the electrodes 20 are connected together by a connector 320. While a pattern of the connector is illustrated as a line or a shape of “ [symbol] ” in the figure, it is not limited thereto, but may assume a diversity of patterns. Further, the connector 320 may be of any type so long as it is conductive and is used in the field of the art.

According to the present invention, the metal wire of an electrode for stimulating cranial nerves can assume a diversity of shapes, provided the shapes can be easily inserted. The electrode can employ a machining method such as mechanical polishing, welding, or the like, and can be additionally machined with ease. Further, the electrode can ensure the micro tube using a simple process so as to supply drugs to the nerve system at low cost.

Although a preferred embodiment of the present invention has been described for illustrative purposes, those skilled in the art will appreciate that various modifications, additions and substitutions are possible, without departing from the scope and spirit of the invention as disclosed in the accompanying claims.

What is claimed is:

1. An electrode for stimulating cranial nerves comprising:
a glass fiber having first and second holes; and
a metal wire disposed in the first hole.

2. The electrode for stimulating cranial nerves according to claim 1, wherein the first and second holes have a diameter in the range of 20 μm to 100 μm.

3. The electrode for stimulating cranial nerves according to claim 1, wherein the electrode has a diameter in the range of 100 μm to 290 μm.

4. The electrode for stimulating cranial nerves according to claim 1, wherein the glass fiber and the metal wire are fixed together using heat- or light-curable polymer.

5. The electrode for stimulating cranial nerves according to claim 1, wherein the glass fiber further includes one or more holes in addition to the first and second holes.

6. A substrate comprising:
a glass substrate having at least one electrode insertion area;
electrodes each being disposed in the electrode insertion area and including a glass fiber having first and second holes, and a metal wire disposed in the first hole; and
a connector connecting the glass substrate and the electrodes together.
